(19) United States
(12) Patent Application Publication
(10) Pub. No.: US 2026/0053392 A1
(43) Pub. Date: Feb. 26, 2026

(54) A SENSOR ARRANGEMENT AND A SYSTEM FOR MONITORING PEOPLE

(71) Applicant: MariElectronics Oy, Vantaa (FI)

(72) Inventor: Göran SUNDHOLM, Tuusula (FI)

(73) Assignee: MariElectronics Oy, Vantaa (FI)

(21) Appl. No.: 19/105,671

(22) PCT Filed: Aug. 17, 2023
(86) PCT No.: PCT/FI2023/050472

§ 371 Date: Feb. 21, 2025

(30) Foreign Application Priority Data
Aug. 24, 2022 (FI) 20225746

(51) Int. Cl. A61B 5/11; A61B 5/00; A61B 5/05; G01S 13/58; G01S 13/89

(52) U.S. Cl. CPC A61B 5/1126; A61B 5/05; A61B 5/1113; A61B 5/6889; G01S 13/584; G01S 13/89

(57) ABSTRACT

A system and a sensor arrangement for observing presence, location, movement and/or attitude of a person in a monitored area. The sensor arrangement includes at least one means for processing the measurement signal of the sensor, such as measuring electronics, and at least one means for communicating measurement results and/or data relating to the measurement results for further processing. The sensor arrangement includes at least two sensors which are configured to measure to different directions from each other. The at least two sensors are radar-based sensors, such as frequency-modulated continuous-wave MIMO radar-based sensors, configured to detect persons in the monitored area, and measure and detect movement, location, velocity, route and/or shape of the monitored person.

### FIELD OF THE INVENTION

The invention relates to a sensor arrangement and a system for monitoring people.

### BACKGROUND OF THE INVENTION

Monitoring people is useful in many situations. E.g. monitoring of the condition of elderly people in a home environment is important, if it is desired to lengthen the possibility of an aging population coping in their home environment. Safety bracelet systems are nowadays widely used for these kinds of applications. Their weakness is that the user must wear the bracelet continuously and must be able to press the alarm button in an emergency. There are also bracelets that check the state of health of the user, but they have the same problems as described above, and additionally, there are further problems with false alarms.

There have also been presented solutions, in which a film of piezoelectric material is installed on the floor, in which the film registers pressure changes caused by movement on the surface of the floor. Also known in the prior art is the use of sensors to be installed on the floor, or under it, that detects presence and movements of people, without requiring a change in pressure, but functions by means of capacitive sensors.

The possibility of using video cameras, movement detectors that are based e.g. on detecting infrared light, or e.g. ultrasound sensors, for monitoring the condition and state of elderly people is also presented in prior art. For example, WO2012164169 document discloses a method and a system that are based on ultrasound technology for tracking objects.

Monitoring can be also important in a retail environment to see how customers behave e.g. in a retail store and what products they are buying. Also, some systems are presented in the prior art which can be used to monitor people in the retail store environment, but these separate prior art solutions are difficult to install and integrate to other system and they are also not able to provide reliable information on customer behavior.

Some prior art solutions are known, which use millimeterwave (MMW) radar for tracking persons.

A drawback of observation and monitoring systems, known in the prior art, is that they are not able to provide reliable measurement results in different kinds of circumstances. The prior art systems are also difficult to install and maintain.

### BRIEF DESCRIPTION OF THE INVENTION

The purpose of the solution according to the invention is to eliminate the problems of prior art. The system according to the invention is characterized by what is stated in the characterization part of claim 1. The system according to the invention is characterized by what is stated in the characterization part of claims 2-14 relating to the system. The system according to the invention is also characterized by what is stated in the characterization part of claim 15 relating to the system.

According to a first aspect, the invention relates to a sensor arrangement for observing presence, location, movement and/or attitude of a person in a monitored area. The sensor arrangement comprises at least one means for processing the measurement signal of the sensor, such as measuring electronics, and a means for communicating measurement results and/or data relating to the measurement results for further processing. The sensor arrangement comprises at least two sensors which are configured to measure to different directions from each other, wherein the at least two sensors are radar-based sensors, such as frequency-modulated continuous-wave MIMO radar-based sensors, configured to detect persons in the monitored area, and to measure and detect movement, location, velocity, route and/or shape of the monitored person.

In one embodiment of the invention the at least two radar-based sensors are arranged in connection with a structure and/or body of the sensor arrangement.

In one embodiment of the invention the arrangement comprises three radar-type sensors which are arranged so that a center line of the measurement zone of each sensor is arranged to be essentially 120 degrees to the center lines of next sensors.

In one embodiment of the invention the arrangement comprises four radar-type sensors which are arranged so that a center line of the measurement zone of each sensor is arranged to be essentially 90 degrees to the center lines of next nearby sensors.

In one embodiment of the invention, one means for processing the measurement signal of the sensor is arranged for each sensor of the at least two sensors.

In one embodiment of the invention, one means for processing the measurement signal of the sensor is arranged and connected to all sensors of the sensor arrangement.

In one embodiment of the invention the sensor arrangement comprises at least one speaker.

In one embodiment of the invention the sensor arrangement comprises at least one light source for providing emergency lighting.

In one embodiment of the invention the sensor arrangement comprises attachment means with which the sensor arrangement is attachable to an opening.

In one embodiment of the invention the sensor arrangement and/or each sensor is configured to monitor health related functions of the person, such as breathing frequency and heart rate.

In one embodiment of the invention the sensor arrangement and/or each sensor comprises a first operating mode and a second operating mode, wherein, in the first operating mode the sensor is configured to track movement of the monitored person, and in the second operating mode the sensor is configured to measure and/or further analyze measurements relating to a part of the monitored area, in which movement of a person was observed in the first operating mode in order to observe health related functions of the person.

In one embodiment of the invention the sensor arrangement or each sensor is configured to analyze the measurement signal in such a way that the phase of the measurement signal is determined, e.g. in the second operating mode, in order to observe movement of the person, such as heartbeat and/or breathing of the person.

In one embodiment of the invention the at least two sensors are radar sensor configured to observe the elevation, azimuth, movement and/or distance of objects with continuous-wave radar technique, such as a frequency-modulated continuous-wave (FMCW) and/or wherein the system or sensor arrangement is configured to create point cloud data from the information received from radar sensors for the monitored area by combining information from multiple sensors.

In one embodiment of the invention the sensor arrangement comprises a means for detecting the orientation of the sensor arrangement, such as an acceleration sensor, and the sensor arrangement is configured to take the detected orientation of the sensor arrangement into account when determining measurement results for the monitored person, e.g. by compensating the measurement results based on the detected orientation.

According to a second aspect, the invention relates to a system for observing presence, location, movement and/or attitude of one or more objects in a monitored area. The system comprises at least one sensor arrangement according to any embodiment of the invention wherein the sensor arrangement or sensor arrangements are fitted in the monitored area, e.g. on ceiling, wall and/or a stand.

With the solution of the invention, it's possible to obtain valuable information about persons in the monitored area. With the sensor arrangement of the invention bigger spacing of the installed sensors can be achieved because each sensor arrangement comprises multiple sensors. This has the advantage of an easier installation, cabling and maintenance of the system when compared to separate sensors as there does not have to be so many individual sensors to achieve good coverage. As the sensors of the sensor arrangement are radar-based sensors monitoring of the people can be done in an unobtrusive manner, e.g. when compared to cameras.

Various other advantages will become clear to a skilled person based on the following detailed description.

The expression “a plurality of” refers herein to any positive integer starting from two, e.g. to two, three, or four. The terms “first”, “second” and “third” do not denote any order, quantity, or importance, but rather are used to distinguish one element from another.

The exemplary embodiments of the present invention presented herein are not to be interpreted to pose limitations to the applicability of the appended claims. The verb “to comprise” is used herein as an open limitation that does not exclude the existence of also un-recited features. The features recited in dependent claims are mutually freely combinable unless otherwise explicitly stated.

The novel features which are considered as characteristic of the present invention are set forth in particular in the appended claims. The present invention itself, however, both as to its construction and its method of operation, together with additional objectives and advantages thereof, will be best understood from the following description of specific embodiments when read in connection with the accompanying drawings.

### BRIEF DESCRIPTION OF THE FIGURES

In the following, the invention will be described in more detail by the aid some embodiments with reference to the drawings 1-4, wherein

FIGS. 1A-1F presents a diagrammatic and simplified sensor arrangement according to one embodiment of the invention,

FIG. 2A-2E presents a diagrammatic and simplified sensor arrangement according to one embodiment of the invention,

FIG. 3A-3F presents a diagrammatic and simplified sensor arrangement according to one embodiment of the invention, and

FIG. 4 presents in a simplified manner one example embodiment related to arranging at least part of the solution of the invention to an area which is monitored.

### DETAILED DESCRIPTION OF THE INVENTION

In the solution of the invention data from person moving in the monitored area is collected with radar-based sensor. The monitored area can be for example a store, a cruise ship, a hotel, a shopping mall, an office or an apparent, a home. The sensor arrangement comprises at least one means for processing the measurement signal of the sensor, such as measuring electronics, and a means for communicating measurement results and/or data relating to the measurement results for further processing. The sensor arrangement comprises at least two radar-based sensors which are configured to measure to different directions from each other.

The sensor arrangement can be used to monitor number of people in the measurement area, locations where people move, where they are stationary and/or how long duration people are stationary at a certain location. It can be determined how long a person is immobile for example in a chair, bed or floor in their home environment. For example, in retail environment it can be determined how long duration customers are examining products before buy-decision, e.g. how long they spend time stationary beside certain product that has been determined to have been purchased.

In one embodiment of the invention multiple, e.g. four, radar based sensors are arranged in connection with each other, for example in one enclosure and so that each of the sensors measures to different direction from each other. In one embodiment the sensor arrangement is configured to measure 360 degrees. In the example of four radar sensors, each sensor measures 90-degree area. In the example of three radar sensors, each sensor measures 120-degree area. The sensor arrangement can be installed e.g. in the ceiling of the monitored area.

The sensor arrangement and/or a system can be configured to receive measurement information from every sensor and create a point cloud data of the monitored area by combining measurement data from all radar type sensors.

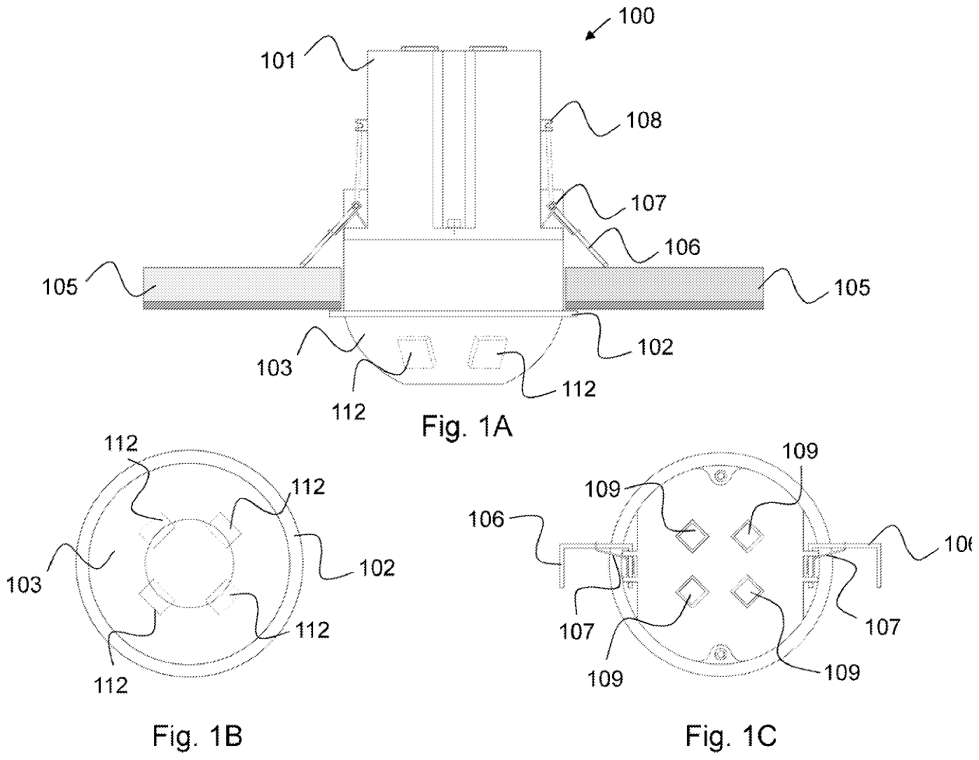

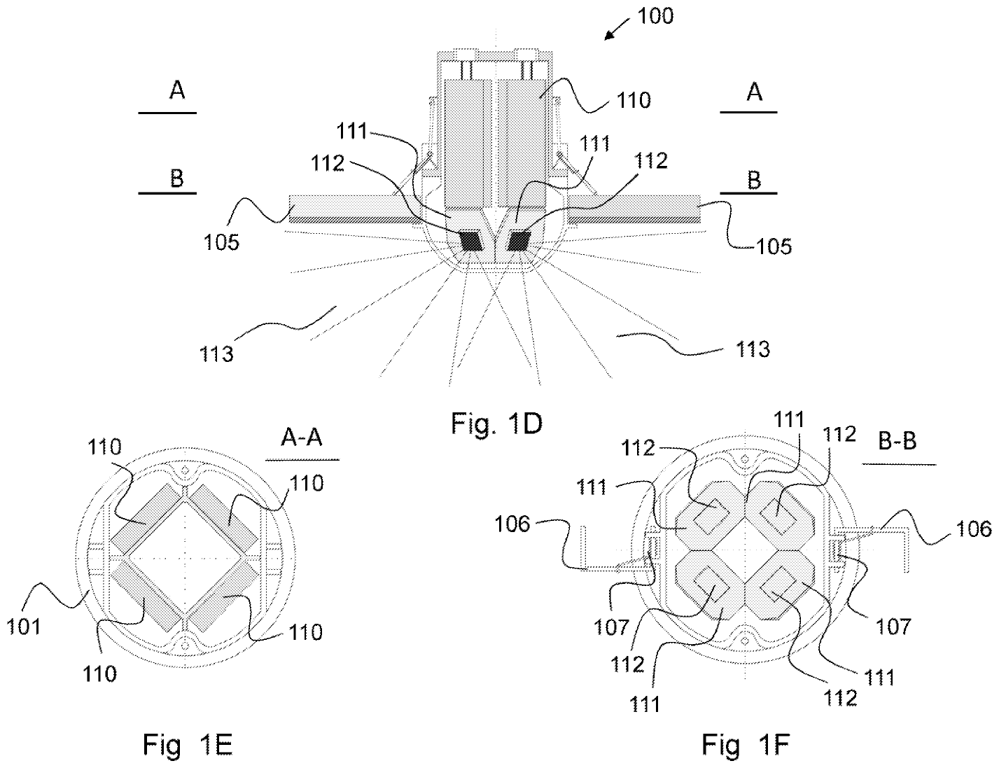

FIGS. 1A-1F presents in a diagrammatic and simplified manner a sensor arrangement 100 presents a diagrammatic and simplified sensor arrangement according to one embodiment of the invention. In this embodiment the sensor arrangement comprises four radar sensors. The sensor arrangement comprises a base part 101 inside which at least part of the components of the sensor arrangement are arranged. FIG. 1A presents the sensor arrangement from a side view when the sensor arrangement is installed to an opening in a ceiling structure, FIG. 1B presents the sensor arrangement from below and FIG. 1C presents the sensor arrangement from above. The base part of the sensor arrangement can comprise locking means 106 with which the sensor arrangement can be attached to the structure, such as an opening in the ceiling 105. In one embodiment of the invention the locking means can be turned away from the locking position to the side of the base part so that the sensor arrangement can be pushed through the opening e.g. in the ceiling. The base part can comprise attachment points 108 for the locking means to which the locking means can be turned and attached, e.g. during transportation and stocking. The arrangement can comprise springs 107 which open the locking means and keep them in use position, i.e. in a position in which the keep the arrangement fastened to the ceiling structure. The sensor arrangement can also comprise an edge part 102 which is arranged to be wider the upper part of the base part so that the edge part will prevent the sensor arrangement to go through the opening, e.g. in the ceiling. The sensor antennas 112 or antenna arrays can be covered with a cover part 103. On the top of the sensor arrangement there can be connectors 109 or openings for cables with which the sensor arrangement can be electrically and/or communicatively connected to electrical and/or communication network.

FIGS. 1D, 1E and 1F present the sensor arrangement as a cut away drawing FIG. 1E presenting a cut away at line A-A of FIG. 1D and FIG. 1F presenting a cut away at line B-B or FIG. 1D. Control circuit boards 110 for each sensor are arranged inside the base part. At least part of means for processing the measurement signal of the sensor, such as measuring electronics, and/or at least one means for communicating measurement results and/or data relating to the measurement results for further processing can be carried out by the control circuit board. Each control circuit board 110 is connected to a radar circuit board 111 to which at least one radar antenna 112 or antenna array is arranged. The control circuit boards 110 are arranged in 90-degree angle to the next control circuit board as presented in the figure. The radar circuit boards 111 and antennas 112 or antenna arrays are also arranged in 90-degree angle to the next radar circuit board and antenna or antenna array. The radar circuit boards, and antennas or antenna arrays can also be angled down to allow an optimal measurement area 113 and monitoring distance for the sensor array.

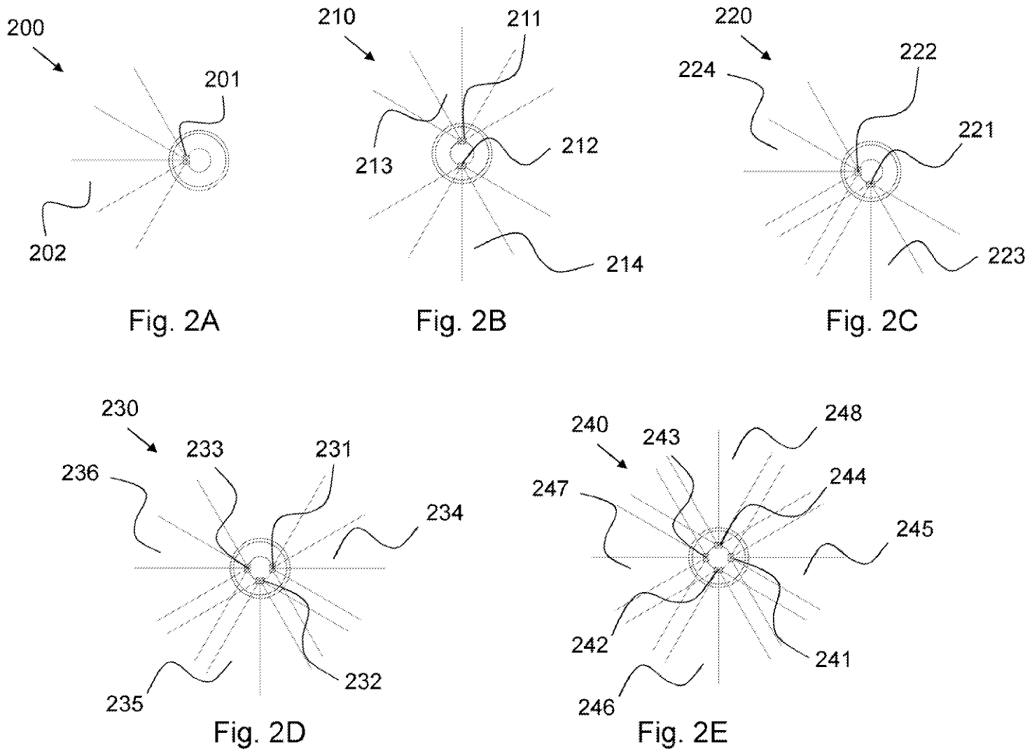

In FIGS. 2A-2E different sensor arrangement configurations are presented. In some embodiments of the invention, it can be selected how many sensors are installed and arranged to the sensor arrangement. In the FIG. 2A one sensor 201 is arranged to the sensor arrangement 200. This kind of sensor arrangement can be used e.g. in a corner of a space when no wide measurement area 202 is needed. FIG. 2B presents a sensor arrangement 210 with two installed sensors 211, 212. In this embodiment the sensors 211, 212 are arranged to measure to opposite directions. This embodiment can be used e.g. in a corridor. FIG. 2C presents a sensor arrangement 220 with two installed sensors 221, 222. In this embodiment the sensors are arranged to measure measurement areas 223, 224 next to each other and in 90-degree angle between the sensors. This embodiment can be used e.g. in a corner and/or a corridor. FIG. 2D presents a sensor arrangement 230 with three installed sensors 231, 232, 233. In this embodiment the sensors are arranged to measure to three different directions and measurement areas, 234, 235, 236. This embodiment can be used e.g. in the edge of the measurement area, e.g. in close to the wall. FIG. 2E presents a sensor arrangement 240 with four installed sensors 241, 242, 243, 244. In this embodiment the sensors are arranged to measure to different directions and measurement areas 245, 246, 247, 248 from each other (and by 90-degree angle from each other). This way the sensor is able to monitor 360-degree area around the sensor.

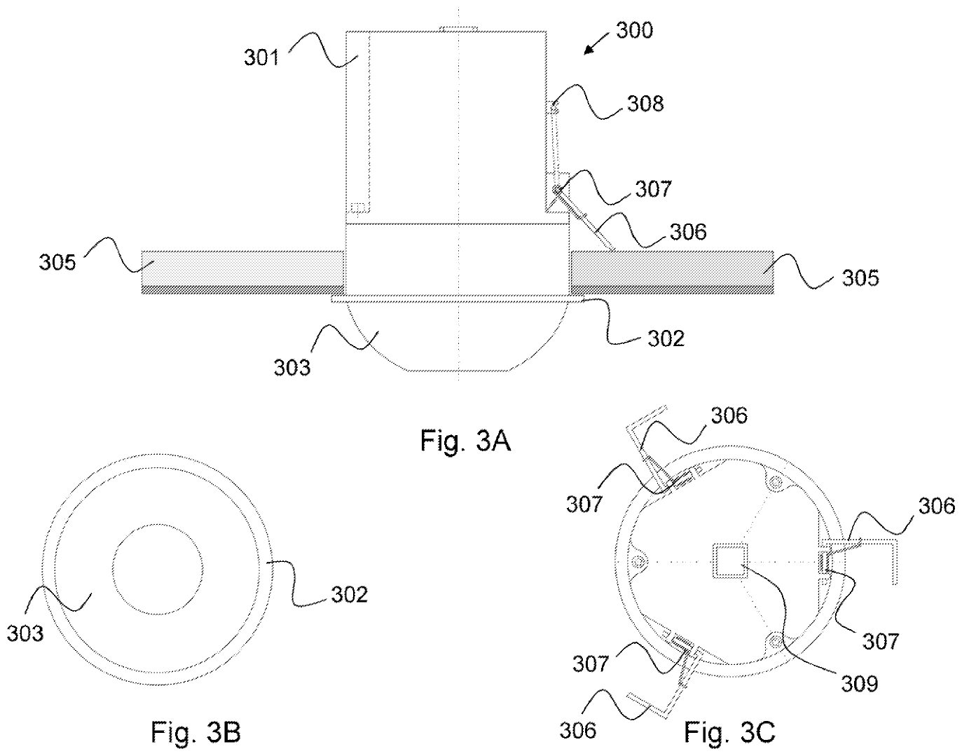

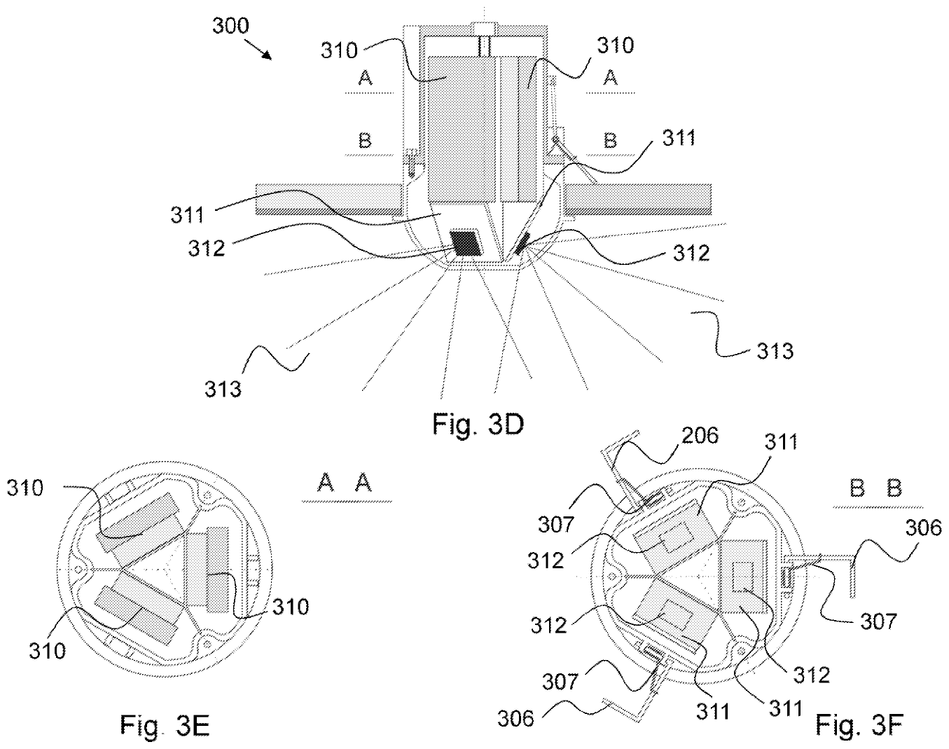

FIGS. 3A-3F present a sensor arrangement which is otherwise similar as the sensor arrangement of FIGS. 1A-1F but instead of four sensors, this embodiment comprises three sensors. The sensor arrangement 300 comprises a base part 301 inside which at least part of the components of the sensor arrangement are arranged. FIG. 3A presents the sensor arrangement from a side view when the sensor arrangement is installed to an opening in a ceiling structure, FIG. 3B presents the sensor arrangement from below and FIG. 3C presents the sensor arrangement from above. The base part of the sensor arrangement can comprise locking means 306 with which the sensor arrangement can be attached to the structure, such as an opening in the ceiling 305. In one embodiment of the invention the locking means 306 can be turned away from the locking position to the side of the base part so that the sensor arrangement can be pushed through the opening e.g. in the ceiling. The base part can comprise attachment points 308 for the locking means to which the locking means can be turned and attached, e.g. during transportation and stocking. The arrangement can comprise springs 307 which open the locking means and keep them in use position, i.e. in a position in which the keep the arrangement fastened to the ceiling structure. The sensor arrangement can also comprise an edge part 302 which is arranged to be wider the upper part of the base part so that the edge part will prevent the sensor arrangement to go through the opening, e.g. in the ceiling. The sensor antennas or antenna arrays can be covered with a cover part 303. On the top of the sensor arrangement there can be connectors 309 or openings for cables with which the sensor arrangement can be electrically and/or communicatively connected to electrical and/or communication network.

FIGS. 3D, 3E and 3F present the sensor arrangement as a cut away drawing FIG. 3E presenting a cut away at line A-A of FIG. 3D and FIG. 3F presenting a cut away at line B-B or FIG. 3D. Control circuit boards 310 for each sensor are arranged inside the base part. At least part of means for processing the measurement signal of the sensor, such as measuring electronics, and/or at least one means for communicating measurement results and/or data relating to the measurement results for further processing can be carried out by the control circuit board. Each control circuit board 310 is connected to a radar circuit board 311 to which at least one radar antenna or antenna array is arranged. The control circuit boards 310 are arranged in 120-degree angle to the next control circuit board as presented in the figure. The radar circuit boards 311 and antennas 312 or antenna arrays are also arranged in 120-degree angle to the next radar circuit board 311 and antenna 312 or antenna array. The radar circuit boards and antennas or antenna arrays can also be angled down to allow an optimal measurement area 313 monitoring distance for the sensor array.

In one embodiment of the invention, one radar-type sensor can cover e.g. a diameter of 10 m when installed in a corner and e.g. 5 m to 7 m when installed downwards from the ceiling, depending on the height. With the sensor arrangement of the invention the measurement area can cover e.g. 20 m as there are multiple sensors with are measuring to different directions.

In one embodiment of the invention the sensor arrangement comprises at least one speaker.

In one embodiment of the invention the sensor arrangement comprises at least one light source for providing emergency lighting.

In one embodiment of the invention the sensor arrangement comprises attachment means with which the sensor arrangement is attachable to an opening.

In one embodiment of the invention, one means for processing the measurement signal of the sensor, such as a control circuit board, is arranged for each sensor of the at least two sensors. In this case the means for processing the measurement signal of the sensor collects or receives data from only one sensors of the sensor arrangement and can analyze it and/or send it to a further unit. This way the sensors of the sensor arrangement can operate independently of each other.

In one embodiment of the invention, one means for processing the measurement signal of the sensor, such as a control circuit board, is arranged and connected to all sensors of the sensor arrangement. In this case one means for processing the measurement signal of the sensor collects or receives data from all sensors of the sensor arrangement and can analyze it and/or send it to a further unit. In one embodiment of the invention the means for processing the measurement signal of the sensor can change the controlled sensor in turns, e.g. by switching the controlled sensor every 1 seconds. In this case, information about the movement is obtained every 3-4 seconds if there are 3-4 sensors in the sensor arrangement.

In one embodiment of the invention the sensor arrangement and/or each sensor is configured to monitor health related functions of the person, such as breathing frequency and heart rate. In one embodiment of the invention the sensor arrangement and/or each sensor comprises a first operating mode and a second operating mode, wherein, in the first operating mode the sensor is configured to track movement of the monitored person, and in the second operating mode the sensor is configured to measure and/or further analyze measurements relating to a part of the monitored area, in which movement of a person was observed in the first operating mode in order to observe health related functions of the person.

In one embodiment of the invention different sensors of the sensor arrangement can be in different operating states. This makes it for example possible that some of the sensors are used in the first operating mode and some sensors are used in second operating mode. The sensors can change the operating modes based on the needs of the system.

In one embodiment of the invention the sensor arrangement or each sensor is configured to analyze the measurement signal in such a way that the phase of the measurement signal is determined, e.g. in the second operating mode, in order to observe movement of the person, such as heartbeat and/or breathing of the person.

In one embodiment of the invention the at least two sensors are radar sensor configured to observe the elevation, azimuth, movement and/or distance of objects with continuous-wave radar technique, such as a frequency-modulated continuous-wave (FMCW) and/or wherein the system or sensor arrangement is configured to create point cloud data from the information received from radar sensors for the monitored area by combining information from multiple sensors.

In one embodiment of the invention the sensor arrangement comprises a means for detecting the orientation of the sensor arrangement, such as an acceleration sensor, and the sensor arrangement is configured to take the detected orientation of the sensor arrangement into account when determining measurement results for the monitored person, e.g. by compensating the measurement results based on the detected orientation.

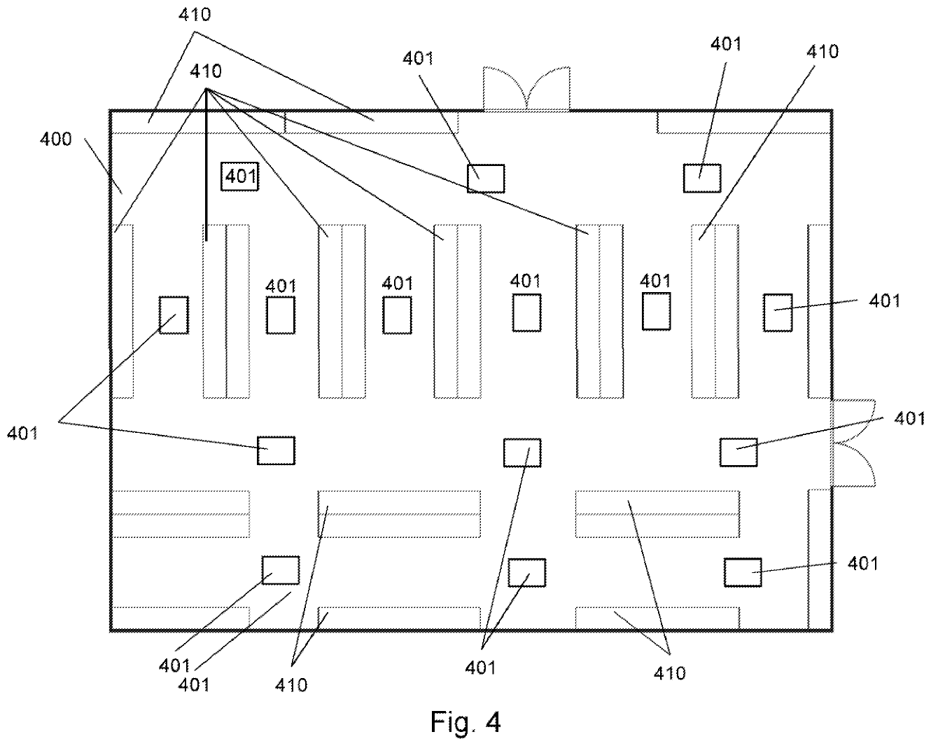

FIG. 4 presents in a simplified manner one example embodiment related to arranging the sensor arrangements in a space which is to be monitored. In this example embodiment the sensor arrangements are arranged to a store and in the FIG. 4 the store is viewed from above. The sensor arrangements 401 are arranged above the aisles between the shelves 410 of the store so that the sensor arrangements are able to measure areas between the shelves where people are moving. In this example the measurement area of all the sensor arrangements 401 arranged in the store covers essentially all areas 400 where people can be moving. The sensors can be arranged so that they are directed directly downwards or slightly tilted.

In one embodiment of the invention, the sensor arrangement can be used to detect and/or give a signal if a sprinkler has been triggered in the monitored space.

In one embodiment of the invention in retail environment, the system can comprise an electronic price label system or be connected to an electronic price label system. The electronic price label base stations can be configured to locate the position of an electronic price label by measuring it with different base stations from different directions. The system can have a direction of arrival detection mode in which it can determine the location of the electronic price label by combining the information of the measured direction of the label from different base stations, e.g. by triangulation. With this solution the system is able to collect locations and/or routes of the electronic price labels. In the direction of arrival detection mode, the base station can send the electronic price labels a command to send signal to the base station, e.g. a Constant Tone Extension (CTE) signal. The base station can comprise an antenna array and the direction of arrival of the signal from the electronic price label can be determined from the antenna array of the base station. With e.g. three base stations, the location of the label can be determined.

In one embodiment, the base station of the electronic price label system can be arranged in same location and/or within same structure as the radar-based sensor or sensors. This way the installation to the store environment is easy as the wiring and installation has to be done in one place for means for detecting movement and base station. With this solution the system is able to collect locations and/or routes of the persons in the store and also locations and/or routes of the electronic price labels. This arrangement can be installed e.g. in the ceiling of the monitored area.

With the sensors used in the solution of the invention a “heat map” type of image at different times can be provided from which it can be seen how often or how many people have been moving in a certain time in certain areas. This information can be used for example for targeted marketing and pricing. In one embodiment of the invention the means for detecting movement, presence and/or location of a person are used to determine that a certain product is taken from the shelf and/or put back to shelf, and/or this way heat mapping data of a certain shelf can be firmed and/or provided.

In one embodiment of the invention the means for detecting movement, presence and/or location of a person can determine fill ratio of at least one shelf or part of a shelf and in another embodiment fill ration can be determined with an additional sensor, e.g. a planar sensor arranged to the shelf or a sensor, such as a camera or distance measuring sensor, arranged for example to the opposite side of the aisle.

In the solution of the invention POS system can provide information on which products have been purchased, at what time and from the electronic price label system or shelf map where each product is placed in the store. Combining this information and using time constraints (delay in taking the product off the shelf until it is at the checkout) will make it possible to form an estimate of the purchase information related to certain products.

In one embodiment of the invention the method comprises sending a message to a mobile device of a person at a certain location based on the determination of presence of the person at the location.

In one embodiment of the invention the system, e.g. an electronic price label system can comprise a routing programs based on a shopping list and store map. The route determined based on the means to detect movement, presence and/or location of a person can be used to compare which products cause the customers to deviate from the planned route and planned products of the shopping list. This information can be used in targeted marketing and pricing, e.g. at the store. In one embodiment of the invention monitoring movement of a person comprises determining a route used by a person in the store with means for detecting movement, presence and/or location of a person.

In a case where the store comprises a sensor network comprising multiple sensors customer movement can be monitored from one sensor to another and/or the route traveled by an individual customer can be stored and compared. The motion observations generated by certain type of sensor, e.g. the radar-based sensor, can be supplemented with other motion tracking systems such as cameras, PIR sensors, etc.

The processor, central unit and/or measuring electronics used in the solution of the invention can be integrated into the sensors or they can be disposed separately or in separate units. In an embodiment of the invention, with a software executed by the processor the sensor or system can interpret the movements observed with at least one sensor and can give an alarm if the alarm conditions defined for the program are fulfilled.

In one embodiment of the invention only some of the sensors of the area to be monitored have the functionality enabling the issuing of an alarm signal as described above. For example, the sensors in only some rooms, such as in the living room, can be provided with this functionality and the sensors in other rooms send a notification onwards immediately after a fall is detected and/or measurement results of a measured person are not at acceptable and/or in predefined range. In one embodiment of the invention only some of the sensors in one space, such as in a room, comprise the functionality enabling the issuing of an alarm signal as described above.

The system can also comprise a control center and the predetermined information concerning presence, location, movement and/or attitude of the object can be sent to the control center. The alarm terms used by the system can be changed, e.g. on the basis of presence information, which can be e.g. received from an RFID reader. A notification can be sent or an alarm can be given e.g. to an external alarm system or wirelessly to a central server of the system, from which server the alarm is directed onwards.

The system can also have a memory means, in which the system is adapted to record a measurement signal, or information derived from it, for observing the chronological dependency of the area being monitored and the behavior of objects. By means of this the system can give an alarm e.g. if a person being monitored has not got out of bed or visited the kitchen for a certain time, or if the person has gone to the toilet too often or if the vital functions of the observed person, such as breathing or heartbeat, have changed during time. The memory means also enables learning of a more common daily rhythm and the detection of aberrations occurring in it.

In the following one example embodiment is described. In this example embodiment, first operating mode of the sensor is used to track presence and movements of people, e.g. in a single room. In this embodiment the tracking is carried out with the measured point cloud data. Doppler range needed is given by

$${f}_{D max} = \frac{2 v}{λ}$$

In one example, if the person is moving with a speed of 1 m/s, the needed Doppler range is +−40 Hz and the maximum measurement interval is 25 ms at 60 GHz frequency. Inbreathing lasts about 2 seconds. If the corresponding movement is 5 cm, the Doppler range needed is +−1 Hz and the sweeping time is one second.

When the system observed that the person has stopped, it can activate the second operating mode, in which it is able to track vital functions of the person, such as heartbeat and/or breathing, such as breathing interruptions and/or frequency.

After the vital functions of the object are determined, the system can deactivate the second operating mode. In one example embodiment, the system can determine vital function of the same person periodically, e.g. as long as the person stays stationary. If the system observes stationary objects, it's starts to determine vital functions of these objects by using the second operating mode.

In one example embodiment of the invention operation in the second operating mode can be implemented for example so that when the stationary object has been detected, point cloud data around an area of the detected object is saved and analyzed. The saved packages can be generated periodically, e.g. every 600 ms. In one embodiment of the invention the data can be transferred to central control units for analysis. With the analysis of the signal, i.e. the point cloud data, information about small movements of the object can be observed and thus the system is able to determine e.g. breathing activity and/or heartbeat of the person.

In one embodiment of the invention the sweep time of the sensor is longer in the second operating mode and because of this better signal to noise ratio can be achieved. Also, more TX-antennas can be utilized because there is more time available for measurement. This way the angle resolution can be improved. For improving the distance resolution, the frequency sweep range can be increased.

The doppler frequency can be determined e.g. with Fast Fourier Transformation (FFT). The vital function activity, e.g. heartbeat and breathing activity, can be determined based on the determined doppler frequency. In one embodiment of the invention there are more TX antennas used in the second operating mode to increase the spatial resolution. Signal processing can be done for a smaller area because the measured person is not moving.

In one embodiment of the invention the sensor can comprise a radio-based identification means for identifying a person. The radio-based identification means can be for example Bluetooth, Bluetooth low energy (BLE) or Zigbee based means. In this embodiment the system can recognize the object and a radio-based device carried by the object, such as a bracelet, a watch, a mobile device, a tag, and the measurement results can be linked to the specific recognized person. This way the systems is able to know who is present in the measured area and to whom the measurement results relate.

In one embodiment of the invention the radio-based identification means can comprise an antenna array that makes it possible to more accurately associate the identification devices to their carriers when there are more than one person and device present.

In one embodiment of the invention the alarms can be automatically disabled if the identification means detect a certain person such as a nurse in the monitored area.

In one embodiment of the invention the alarm conditions of the system can include the identity of the person. For example, an alarm can be triggered when an unauthorized person enters certain location.

In one embodiment of the invention the radio-based identification means, for example Bluetooth, Bluetooth low energy (BLE) or Zigbee based means, can be used in locating a person or assist in locating the person. The sensor can include several antennas for radio-based identification means, e.g. Bluetooth, BLE or Zigbee antennas to enable direction finding techniques, for example Zigbee, Bluetooth or Bluetooth low energy (BLE) direction finding techniques, e.g. according to Bluetooth 5.1 specification. In one embodiment of the invention, if the radar of the sensor detects movement but the radio-based identification means do not detect a remotely readable tag or device, such as a Bluetooth, BLE or Zigbee tag or device, then the person detected by the radar can be considered a visitor. If, on the other hand, the radar detects a remotely readable tag or device, such as a Bluetooth, BLE or Zigbee tag or device, then the detected person can be identified, and actions can be taken based on the identified person. In one example embodiment, when a resident is in a room and there is also an assisting person, the status of the person or the room can be set in the system to “an assisting person present in the room”.

In the same way, an alarm made by a resident can also be acknowledged as the system recognizes that a person, who is not a resident in the room, enters the room. In this case the alarm can be acknowledged automatically. In one embodiment an alarm is not acknowledged automatically but requires an active identifiable event, e.g. from the user device.

In one embodiment of the invention identification of the detected person can be done with other means, for example with surveillance cameras, e.g. arranged to corridors. In this case the radar-based sensor detects that someone is entering the room and the system can check information from the surveillance cameras, e.g. from a certain point in time from the surveillance recording, in which a person can be seen to enter the room. In one embodiment this recording could be linked to the room as an entry event and the entrant could be identified later, if necessary, by looking at the recording. In that case the identification can be automatic but automatic identification doesn't have to be implemented if not preferred. If automatic identification from the video is used, it can be implemented e.g. based on facial recognition techniques. In one embodiment, if user can be identified in other ways, facial recognition or video-based recognition is not used. In one embodiment video-based identification is only used if person can't be identified in any other way.

In one embodiment of the invention the measurements and observations collected by the sensor arrangement of the invention can be utilized in crowd control management. In this case people can be guided in the monitored area to the best available routes, elevators, etc., e.g. based on the number of people in the area. This kind of solution can be used e.g. in hotels, shops, shopping malls, offices and cruise ships. Guiding can be implemented for example using light strips and/or guiding displays which can guide people dynamically.

In one embodiment of the invention the sensor arrangement or information provided by the sensor arrangement can be used in controlling elevators of an elevator system. In this case the sensor means can be arranged e.g. to a waiting area of an elevator, for example on each floor, and/or to the elevator car. The sensor arrangement can be used to give information about the presence and number of passengers waiting for an elevator car at least in the waiting area and/or in the elevator car. The movement of the elevator cars of the elevator system can be controlled based on the information received from the sensor arrangement or sensor arrangements. A sensor arrangement comprising 1-4 sensors can be used in this embodiment, e.g. based on the size of the monitored area. E.g. in one example embodiment, a sensor arrangement with one sensor can be used in an elevator car.

In one embodiment of the invention people can be guided to available elevators based on guiding means described above, e.g. by light strips and/or guiding displays which can guide people dynamically.

The embodiments of the invention described herein before in association with the figures presented and the summary of the invention may be used in any combination with each other. At least two of the embodiments may be combined together to form a further embodiment of the invention.

The specific examples provided in the description given above should not be construed as limiting the applicability and/or the interpretation of the appended claims. Lists and groups of examples provided in the description given above are not exhaustive unless otherwise explicitly stated.

1. A sensor arrangement for observing presence, location, movement and/or attitude of a person in a monitored area, wherein
the sensor arrangement comprises at least one means for processing the measurement signal of the sensor, such as measuring electronics, and at least one means for communicating measurement results and/or data relating to the measurement results for further processing,
wherein the sensor arrangement comprises at least two sensors which are configured to measure to different directions from each other, and
wherein the at least two sensors are radar-based sensors, such as frequency-modulated continuous-wave MIMO radar-based sensors, configured to detect persons in the monitored area and measure and detect movement, location, velocity, route and/or shape of the monitored person.

2. The sensor arrangement according to claim 1, wherein the at least two radar-based sensors are arranged in connection with a structure and/or body of the sensor arrangement.

3. The sensor arrangement according to claim 1, wherein the arrangement comprises three radar-type sensors which are arranged so that a center line of the measurement zone of each sensor is arranged to be essentially 120 degrees to the center lines of next sensors.

4. The sensor arrangement according to claim 1, wherein the arrangement comprises four radar-type sensors which are arranged so that a center line of the measurement zone of each sensor is arranged to be essentially 90 degrees to the center lines of next nearby sensors.

5. The sensor arrangement according to claim 1, wherein one means for processing the measurement signal of the sensor is arranged for each sensor of the at least two sensors.

6. The sensor arrangement according to claim 1, wherein one means for processing the measurement signal of the sensor is arranged and connected to all sensors of the sensor arrangement.

7. The sensor arrangement according to claim 1, wherein the sensor arrangement comprises at least one speaker.

8. The sensor arrangement according to claim 1, wherein the sensor arrangement comprises at least one light source for providing emergency lighting.

9. The sensor arrangement according to claim 1, wherein the sensor arrangement comprises attachment means with which the sensor arrangement is attachable to an opening.

10. The sensor arrangement according to claim 1, wherein the sensor arrangement and/or each sensor is configured to monitor health related functions of the person, such as breathing frequency and heart rate.

11. The sensor arrangement according to claim 1, wherein the sensor arrangement and/or each sensor comprises a first operating mode and a second operating mode,
wherein in the first operating mode the sensor is configured to track movement of the monitored person, and in the second operating mode the sensor is configured to measure and/or further analyze measurements relating to a part of the monitored area, in which movement of a person was observed in the first operating mode in order to observe health related functions of the person.

12. The sensor arrangement according to claim 1, wherein the sensor arrangement or each sensor is configured to analyze the measurement signal in such a way that the phase of the measurement signal is determined, e.g. in the second operating mode, in order to observe movement of the person, such as heartbeat and/or breathing of the person.

13. The sensor arrangement, according to claim 1, wherein the at least two sensors are radar sensor configured to observe the elevation, azimuth, movement and/or distance of objects with continuous-wave radar technique, such as a frequency-modulated continuous-wave (FMCW) and/or wherein the system or sensor arrangement is configured to create point cloud data from the information received from radar sensors for the monitored area by combining information from multiple sensors.

14. The sensor arrangement, according to claim 1, wherein the sensor arrangement comprises a means for detecting the orientation of the sensor arrangement, such as an acceleration sensor, and the sensor arrangement is configured to take the detected orientation of the sensor arrangement into account when determining measurement results for the monitored person, e.g. by compensating the measurement results based on the detected orientation.

15. A system for observing presence, location, movement and/or attitude of one or more objects in a monitored area,
wherein the system comprises at least one sensor arrangement according to claim 1,
wherein the sensor arrangement or sensor arrangements are fitted in the monitored area, e.g. on ceiling, wall and/or a stand.

16. The sensor arrangement according to claim 2, wherein the arrangement comprises three radar-type sensors which are arranged so that a center line of the measurement zone of each sensor is arranged to be essentially 120 degrees to the center lines of next sensors.

17. The sensor arrangement according to claim 2, wherein the arrangement comprises four radar-type sensors which are arranged so that a center line of the measurement zone of each sensor is arranged to be essentially 90 degrees to the center lines of next nearby sensors.

18. The sensor arrangement according to claim 3, wherein the arrangement comprises four radar-type sensors which are arranged so that a center line of the measurement zone of each sensor is arranged to be essentially 90 degrees to the center lines of next nearby sensors.

19. The sensor arrangement according to claim 2, wherein one means for processing the measurement signal of the sensor is arranged for each sensor of the at least two sensors.

20. The sensor arrangement according to claim 3, wherein one means for processing the measurement signal of the sensor is arranged for each sensor of the at least two sensors.
